(19) United States
(12) Patent Application Publication
(10) Pub. No.: US 2010/0137767 A1
(43) Pub. Date: Jun. 3, 2010

(54) EXTRICATION DEVICE AND METHOD

(76) Inventor: Brian Carlin, Dundee (GB)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/447,404

(22) PCT Filed: Sep. 14, 2007
(86) PCT No.: PCT/GB07/03495

§ 371 Date: Jan. 27, 2010

(30) Foreign Application Priority Data
Oct. 27, 2006 (GB) 0621320.1

(51) Int. Cl. A61F 5/00

(52) U.S. Cl. 602/18

(57) ABSTRACT

A spinal restraint apparatus for use in emergency extrication, where potential back, neck and spinal injuries may exist. The apparatus comprises a collar neck support device and an elongate support member. Further described are methods of using the apparatus.

### FIELD OF THE INVENTION

This invention relates to an apparatus and method of using the same for emergency extrication where potential back, neck and spinal injuries may exist.

### BACKGROUND

Subjects with spinal cord injuries (SCI) present a variety of treatment challenges for emergency/rescue personnel. The main goal of emergency/rescue personnel is to minimise a subject's spinal cord damage following their initial injury, in particular to minimise additional cord damage during improper handling or transportation of the subject to a hospital setting. To minimise cord damage it is known that the spine should be restrained in the neutral position such that the internal area of the spinal canal and the diameter of the cord remains constant. This is typically achieved by restraining movement of the subject's head, neck and spine during extrication/movement of the subject.

Extrication of a subject is typically classified into routine extrication, urgent extrication and emergency extrication depending on the injuries of the subject and the other hazards around the subject.

Routine extrication is deemed to occur when, there is no obvious injury to the subject, but the mechanism of injury (M.O.I.) is such that there is a potential for a spinal cord injury.

Urgent extrication is deemed to occur when along with a potential spinal cord injury, there are or may be other life threatening injuries to the subject and access to the subject is difficult.

Emergency extrication is when life threatening injuries exist, i.e. a blocked airway or when life threatening hazards are present i.e. fire or risk of explosion and the subject must be moved as quickly as possible.

In both routine extrication and urgent extrication, suitable rescue equipment is generally available, such equipment may include, for example, emergency backboards which immobilise the subject's spine, neck and head and allow the subject to be moved without exacerbating the subject's injuries. However, such equipment is relatively time consuming to apply to the subject and thus is generally not used in emergency extrication situations.

Typically, in emergency extrication, the subject is moved without support being provided to the subject to protect the cervical spine area and minimise damage to the spinal cord. For example, in emergency extrication, the subject is typically manually pulled from their location of danger to another location. A specific example, is a road traffic collision when a subject involved in the collision may need to be removed from the vehicle rapidly, for example due to the risk of fire or another danger (serious trauma). The existing procedure is to manually support the subject's head whilst removing the subject through a side door of the vehicle. However, this procedure risks damage to the spinal column and/or cord of the subject.

The provision and fitting of conventional collar devices which typically require to be sized and adjusted to fit, and backboards, as might be used for routine or urgent extrication is typically too time consuming to be used in emergency extrication and thus there is a need for alternative apparatus and methods to achieve emergency extrication with reduced risk of spinal column and/or cord damage to the subject being extricated.

### SUMMARY OF THE INVENTION

In view of the above, there is a need for apparatus or a system which can be used to restrain movement of a subject's head and spine with the aim of protecting the cervical spine area and minimising any damage to the spinal column and/or cord on movement of the subject. In particular, there is a need for apparatus which is suitable for use during emergency extrication of a subject.

The inventor has surprisingly shown that adequate support can be provided to the cervical spinal and/or the head area during emergency extrication using a spinal restraint apparatus, without the need for additional support apparatus.

According to a first aspect of the present invention there is provided a spinal restraint apparatus for emergency extrication comprising;
(i) an elongate flexible support member of length in the range 0.5 m to 5.5 m; and
(ii) a collar neck support device.

The collar device provides support and minimises anterior movement of the head i.e. movement in a forward direction, for example towards the chest, and is useful in preventing and/or exacerbating hyperflexion of the neck. This is advantageous in minimising damage if the subject is suspected of having spinal injury. In use, the collar device is of a length and width that it can extend around a subject's neck under the subject's chin. In preferred embodiments the collar neck support device is flexible such that it can be wrapped around a subject's neck to minimise flexion of the head and forward anterior movement.

In particular embodiments, the collar neck support and elongate support member, when used in combination, support the head and neck within 10 degrees from a neutral position. This minimises the potential risk of damage to the spinal column and/or cord of a subject during extrication of a subject.

In particular embodiments the collar neck support device can be formed from pliable and/or mouldable material such that the collar neck support device is capable of being wrapped around a subject's neck such that it moulds around the neck and fits between the mandible of the jaw and the top of the clavicles where they meet the sternum. The flexibility of the neck support which enables it to be configurable to the contours of the neck, even when the subject is wearing clothing, to provide a supportive fit, is advantageous, as one size of the collar neck device can fit the anatomy of any subject without requiring pre-sizing or shaping of the collar device. This is advantageous over conventional collar devices for controlling movement of the head which are typically rigid and provided in a range of sizes. Typically, to use conventional collar devices a suitable size of collar device has to be selected for a particular subject, which may require anatomical measurement of the subject, and then time is required to pre-size and adjust the conventional rigid collar to provide a supportive fit to the subject to minimise movement of the head.

Flexibility of the collar device of the present invention can be provided by forming the device from pliable and/or mouldable material such that the collar neck support device can adapt to the contours of the clothing and provide suitable support to the subject to minimise the forward movement of the head. However, the material from which the collar device can be formed should, when in use, provide support to the neck of the subject to keep the neck in a neutral or close to a neutral position. In addition, it is advantageous if the material from which collar device is formed provides increased support when the collar device is compressed. In one embodiment the collar device can be formed of a cellular mesh knit, wherein said mesh is arranged into layers of mesh, preferably by folding or rolling, such that the collar device is provided with both strength and resilience in multiple direction. In preferred embodiments the collar device is formed of polyester.

Typically, the initial wrapping of the collar device provides some support to the head and minimises movement of the head. However, in suitable embodiments of the apparatus, the application of the elongate device to the subject, wherein the elongate device overlays at least part of the collar device can ensure, by compression of at least part of the collar device, that tensioning of the elongate device ensures a tight supporting fit of the collar around the head, neck and chest areas of the subject.

Suitably, embodiments of the collar device can be of length in the range 0.4 m to 1.2 m. In particular embodiments, the collar neck support device can be of length in the range 0.6 m to 1.0 m. More preferably the collar device can have a length in the range 0.7 m to 0.9 m, yet more preferably the collar device has a length of 0.8 m.

Suitably the collar device may be formed from flexible material. Alternatively the device may be provided with cut out portions or the like, which allow the device to be wrapped around a subject's neck to mould to the contours of the subject's neck area and to provide a supportive fit to the subject to minimise movement of the subject's head.

In particular embodiments the collar device can comprise a collar support portion interposed between two connecting portions. In such embodiments the collar support portion may be of 30 cm to 50 cm in length and the connecting portions may each be 15 cm to 25 cm in length. In preferred embodiments the collar support portion may be of 40 cm in length and the connecting portions may each be of 20 cm in length.

Functionally, the collar support portion of the collar neck support device can have a width that allows it to extend from the mandible of the jaw to the top of the clavicles where they meet the sternum. Suitably when the collar neck device is in place on a subject, movement of the subject's head toward the chest is minimised.

Suitably in particular embodiments, the collar device can have a width in the range 10 cm to 20 cm. Preferably the collar device has a width of 15 cm.

Suitably in particular embodiments, the collar support device can have a thickness of 1.5 cm to 5 cm.

Suitably in specific embodiments, the collar device can include fastening means, for example, but not limited to, VELCRO™, quick release buckles, or hook and eye catchment means to enable the collar device to be held in position around the neck.

In particular embodiments of the collar device comprising a collar support portion interposed between two connecting portions, the support portion may have a greater thickness than the connecting portions. This can be advantageous as the support portion can resist movement of the head and the connecting portions can be used to secure or locate the collar device around the neck. In particular embodiments the support portion of the collar device may have a thickness in the range 1.5 cm to 5 cm and the connecting portions may have a thickness in the range around 1 mm to 10 mm. This can be advantageous to provide the connecting portions with increased flexibility or pliability with respect to the support portion. Alternatively, it may enable a rescuer applying the apparatus to a subject to achieve a better grip of the collar device such that a more supportive fit can be achieved. In particular embodiments the support portion of the collar device can have a thickness of 1.5 cm to 4 cm and the connecting portions can have a thickness in the range 1 mm to 5 mm.

In particular embodiments of the device which allow the device to be wrapped around the subject's neck and then secured at the side of the neck, for example using fasteners such as VELCRO™, releasable clips or the like, the collar neck device can comprise a side portion, a central support portion and an end portion. In such embodiments one of a side portion and an end portion is shorter than the other, for example at least 50% shorter. In particular embodiments a side portion may be around 4 cm to 15 cm in length, preferably about 7 cm in length, a central support portion may be around of around 30 cm to 50 cm in length, preferably about 40 cm in length and then an end portion may be around 15 cm to 35 cm in length, preferably around 25 cm in length wherein the central portion is interposed between the end portion and the side portion.

In particular embodiments of the device, the collar device can be provided with a width in the range 10 cm to 20 cm. The central portion can have a thickness in the range 1.5 cm to 5 cm and the side and end portions can have a thickness in the range 1 mm to 10 mm. Across a least part of a first surface width of the end portion fastening means, for example VELCRO™, can be provided. In particular embodiments, across at least part of the first width and the opposite width surfaces provided by the collar neck support device there can be provided fastening means, for example VELCRO™. This may be advantageous to allow the device to be fastened at either the left or right hand side of a subject's neck. This is advantageous as it allows a rescuer to approach a subject from either the left or right hand side and may allow the rescuer to be located outside, for example, a car or other space in which the subject is located.

Suitable fastening means to secure the collar neck device around a subject's neck can be provided by VELCRO™.

The collar support may be particularly flexible at the region between the connecting portions and the support portion. This may be advantageous to provide the collar around the neck of a subject. In one embodiment flexibility may be provided in the region between each of the connecting portions and the support portion by reducing the thickness of the collar device at this region.

Suitably in embodiments of the apparatus, the collar neck support device can be a flexible collar, which when in its non-flexed state, is rectangular.

In particular embodiments the collar device may be slideably positionable on the elongate support member, to arrange the collar neck device around the neck of the subject.

In preferred embodiments the collar device is wrapped around a subject's neck and secured in place with fastening means. The wrapping and securing of the collar device suitably locates and positions the collar neck device whilst providing some support to the neck of the subject.

The support member of a collar neck support device may comprise a marker to indicate to a user the portion of the device which, in use, should be placed under the subject's chin. The marker may be provided by any suitable means, for example a label, surface decoration, or a stiffened portion of material.

Suitably in embodiments of the apparatus, the elongate flexible support member can be of length such that in use it is capable of extending around a human subject's torso between a first arm and a torso at a point adjacent to the axilla, over a first shoulder, behind the subject's neck, over a second shoulder and between a second arm and torso of the same human and around the back of the torso. As will be appreciated, the size of the subject will cause variation in the required length of the elongate support member.

Suitably in embodiments of the apparatus, the elongate support member can have a length in the range 1 m to 4 m, for example 3.5 m, more preferably in the range 1.8 m to 3.5 m. In particular embodiments the elongate support member can have a length of about 3 m.

Where the elongate flexible support member is to be used to rescue children it is preferred that it is of length in the range 1 m to 2 m. In particularly preferred embodiments an elongate flexible support member for rescuing a child has a length in the range 1 m to 1.8 m. Suitably in particular embodiments, the elongate flexible member may have a substantially circular or elliptical cross section.

Preferably, the elongate flexible support member is cylindrical in shape.

Suitably in particular embodiments, the elongate flexible member can have a width in the range 4 cm to 15 cm. Preferably the elongate flexible member may have a width in the range 6 cm to 8 cm.

The elongate support member may optionally comprise at least one handle. The provision of a handle(s) on the elongate member may be advantageous to aid gripping and lifting/movement of a subject to whom the elongate member has been applied.

More than two handles may be provided on the elongate member to provide more than two grip points for lifting/movement of the subject.

In particular embodiments the handles may be positioned at the ends of the elongate member to improve the grip on the elongate member provided to a rescuer. However, handles may be provided at various points on the elongate member to accommodate differing sizes of subjects.

Suitably the handle(s) may provide points of attachment for lifting straps. An embodiment of the elongate member may comprise at least one handle, and a flexible collar device slideably attachable to the elongate member.

Suitably, the elongate member is formed from material which allows the elongate member to be arranged around a human subject's torso between a first arm and a torso at a point adjacent to the axilla, over a first shoulder, behind the subject's neck, over a second shoulder and between a second arm and torso of the same human and around the back of the torso.

The elongate support member may comprise a marker to indicate to a user the portion of the member which, in use, should be placed behind the subject's neck. The marker may be provided by any suitable means. In one embodiment a marker may be a strengthened portion of the elongate support member. In a further embodiment, the marker may be printed onto the material of the elongate support member.

Suitably the elongate support portion may comprise a first fastening means, for example a VELCRO™ portion, substantially at an end of the elongate flexible member and a second corresponding fastening means, for example a VELCRO™ portion, spaced apart from the first fixing means and on an opposing side of the elongate flexible member wherein when the elongate flexible member is rolled from the end opposite that on which the first fastening means, for example quick release straps or a VELCRO™ portion(s) is provided, the first and second fastening means, for example a VELCRO™ portion, come into contact with each and hold the elongate flexible portion in a rolled confirmation. This can be advantageous to allow for storage of the elongate member of the apparatus when not in use.

Suitably the elongate portion may include additional or alternative fastening means, for example VELCRO™ portions, to fix to other supports, for example the collar device.

The elongate portion is formed from material which is of suitable strength to carry the weight of a fully grown adult. In preferred embodiments the elongate portion is formed from material which can be arranged such that it can be used to lift a load of at least 150 kg. In specific embodiments the elongate portion can be formed from mesh material. Suitably, in such embodiments said mesh material is arranged to form multiple layers of mesh. For example the mesh can be rolled or folded to form multiple layers. In embodiments the mesh can be made from woven or non-woven materials. The mesh may be formed from textile materials. In examples of the invention, such materials or the filaments to form woven materials can be nylon, polyester, polypropylene or other materials of sufficient strength that when suitably formed into a mesh for use in the elongate member can be used to lift loads of about 150 kg. Suitably, the elongate portion may have a surface formed from a polyurethane coated fabric.

Suitably the spinal restraint apparatus may further comprise a leg support portion.

The leg support portion may be used to secure the movement of the legs, particularly articulation around the hips, such that movement of the legs during extrication and/or transport of the subject causes minimal movement of the restraint device relative to the upper body and neck of the subject and thus minimises movement of the spine.

Suitably in preferred embodiments, the leg support portion can be an elongate flexible member of length in the range 1 m to 5 m. Preferably the leg support can be an elongate flexible support member of length in the range 1 m to 2 m. In particular embodiments the leg support can be 1.8 m in length.

Suitably in embodiments, the leg support portion can comprise fastening means to attach the leg support portion to the elongate support member. Suitable fastening means will be known to those of skill in the art, including, but not limited to buttons, or hooks and eyelets. In a particular embodiment, the fixing means can be provided by VELCRO™. In such an embodiment a hook portion of VELCRO™ may be provided on a leg support portion and a corresponding catch portion of VELCRO™ may be provided on an elongate flexible member.

In preferred embodiments the leg support is substantially circular or elliptical in cross section. Suitably, in particular embodiments, the leg support portion may be cylindrical in shape.

Suitably, in particular embodiments, the leg support portion can have a width in the range 4 cm to 15 cm. Preferably the leg support portion can have a width in the range 6 cm to 8 cm.

The leg support portion can be formed from material which is of suitable strength to carry about half the weight of a fully grown adult. In specific embodiments the elongate portion can be formed from mesh material, wherein said mesh material is arranged to form multiple layers of mesh. For example the mesh can be rolled or folded to form multiple layers. The mesh may be formed from woven or non-woven materials. In particular examples, a mesh can be formed from a knitted textile, wherein said knitted textile when suitably arranged for use in a leg support member, for example by rolling or folding of the mesh material, can be used to lift loads of about 150 kg. Suitably, the leg support member may have a surface formed from a polyurethane coated fabric.

Suitably, the elongate support member, collar device and leg support portion may be constructed of any flexible material, for example, but not limited to, synthetic foam, for example, a closed cell foam, sponge, textiles, plastics or polymers. Suitably, in particular embodiments, the device may be provided with a waterproof/plastic coating. In an embodiment of the apparatus, any one of the elongate support member, collar device and leg support portion may be formed from water resistant material. The spinal restraint apparatus may be formed from durable and waterproof material.

In a particularly preferred embodiment of the apparatus, the components of the apparatus are formed from polyester woven fabric or knitted fabric encapsulated by a polyurethane coated outer fabric.

In particular preferred embodiments the apparatus is formed from mesh. The dimension of the strands and the spaces between the strands can be selected to provide the components of the apparatus with properties selected from high tensile strength, resilience and elasticity.

Forming the components of the apparatus with such material is advantageous as the components of the apparatus can be quickly provided around a subject to be extricated such that they provide support to the subject and minimise movement of the subject's spine and yet have sufficient strength to allow the components to be pulled by a rescuer such that the subject can be lifted out of a confined space, for example a car.

In embodiments the material from which the component parts of the apparatus, in particular the elongate flexible support, can be formed can at least lift a load of 150 kg, preferably a manikin shaped load, around 500 times without adversely affecting the durability of respective apparatus components. In embodiments of components of the apparatus, in particular the elongate flexible support, can withstand up to an extreme load limit of 1.500 kN before its performance deteriorates.

In embodiments of components of the apparatus, in particular the elongate flexible support, a peak load of over 10 kN (i.e. approx. 1,000 kg-force) can be applied to the apparatus component and the apparatus component subjected to 500 lifting times without breaking the component.

The simplicity of design of the spinal restraint apparatus is advantageous as is allows the apparatus to be rapidly applied to a subject to reduce hyperextension/flexion and rotation of the spinal vertebrae during emergency extrication and therefore minimise the likelihood of damage to the spinal cord. In particular embodiments the spinal restraint device may consist of an elongate flexible support member of length in the range 0.5 m to 5.5 m and a collar neck support device.

As will be appreciated from the detailed description, the apparatus of the first aspect of the invention is a system that utilises both the elongate flexible support member and collar neck support device to immobilise and then allow the lifting of a subject to which the apparatus or system is applied. Although the elongate flexible support member and collar neck support device are separate components, and indeed in embodiments wherein a leg support is used as part of the apparatus or system the leg support is a separate component, the component parts act in combination to allow emergency extrication of a subject.

As will be appreciated, in particular embodiments the component parts can be linked to each other or fastened to each other as required.

According to a second aspect of the invention there is provided a method of restricting movement of a subject's head, the method comprising the steps:
(i) providing a collar neck support device to a subject,
(ii) arranging said collar neck support device around the neck of the subject to support the head;
(iii) providing a flexible elongate support member to a subject,
(iv) positioning the flexible elongate support member such that it extends between a first arm and torso of the subject adjacent to an axilla, over the front of the subject's shoulder, behind the subject's neck, over the front of the subject's other shoulder and between the subject's other arm and torso adjacent to the subject's other axilla, and
(v) tensioning the elongate support member to restrict movement of the head of the subject in at least a posterior direction.

By tensioning the elongate support member, the subject's weight is loaded onto the elongate support device which in turn causes the collar support device to further mould to the anatomy of the subject and provide increased support to the patient's head and neck. Suitable support may thus be provided to extricate the subject safely from a first to a second position. The elongate support member, may be advantageously easily positioned around the subject, for example while the subject is trapped in a vehicle, with minimum need for movement of the subject during the application of the elongate support member. This may help in preventing discomfort to the subject, new injuries to the subject, or exacerbating existing injuries.

In one preferred embodiment of the method, in the step of positioning the flexible elongate support member, the elongate support member extends between a first arm and torso of the subject adjacent to an axilla, crosses the subject's chest and passes over the front of the subject's shoulder opposite to the axilla under which the elongate support member extends, passes behind the subject's neck, over the front of the subject's other shoulder, crosses the subject's chest and passes between the subject's other arm and torso adjacent to the subject's other axilla.

In an alternative embodiment of the method, in the step of positioning the elongate support member, the elongate support member is not passed over the subject's chest, but extends between a first arm and torso of the subject adjacent to an axilla, passes over the front of the subject's shoulder above the axilla under which the elongate support member extends, passes behind the subject's neck, over the front of the subject's other shoulder and between the subject's other arm and torso adjacent to the subject's other axilla.

The alternative embodiment may be appropriately used if the subject to which the apparatus is being applied is obese or if the subject has, for example, chest injuries.

Following application of the apparatus to the subject, the subject is typically removed from the site of the injury, for example removed from a vehicle in which the accident occurred.

According to a third aspect of the present invention there is provided the use of the spinal restraint apparatus according to the first aspect of the invention in a method of moving a subject, the method comprising the steps of:
(i) providing a collar neck support device to a subject,
(ii) arranging said collar neck support device around the neck of the subject to support the head;
(iii) providing a flexible elongate support member to a subject,
(iv) positioning the flexible elongate support member such that it extends between a first arm and torso of the subject adjacent to an axilla, over the front of the subject's shoulder, behind the subject's neck, over the front of the subject's other shoulder and between the subject's other arm and torso adjacent to the subject's other axilla,
(v) tensioning the elongate support member to restrict movement of the head of the subject in at least a posterior direction, and
(vi) moving the subject.

In particular embodiments, moving the subject can be movement of the subject out of a confined space, for example a car or a train to another location where they can receive treatment and/or are no longer in danger from a hazard, for example fire.

The method may optionally comprise the step of providing at least one leg support to the subject.

In such a method the leg support is provided around a subject's upper leg, just above the knees. The leg support can be twisted to provide tension to the support such that the knees are suitably held by and immobilised by the leg support. As will be appreciated, the tension should be such that blood flow in subject's leg is not restricted and no damage to the leg(s) is caused.

As will be appreciated, any of the embodiments of the apparatus described in the present application may be used in the methods of the second or third aspects of the invention. For example in one embodiment of the apparatus the collar device may be flexible.

Preferred features and embodiments of each aspect of the invention are as for each of the other aspects mutatis mutandis unless context demands otherwise.

Embodiments of the present invention will now be described, by way of example only, with reference to the accompanying drawings, in which

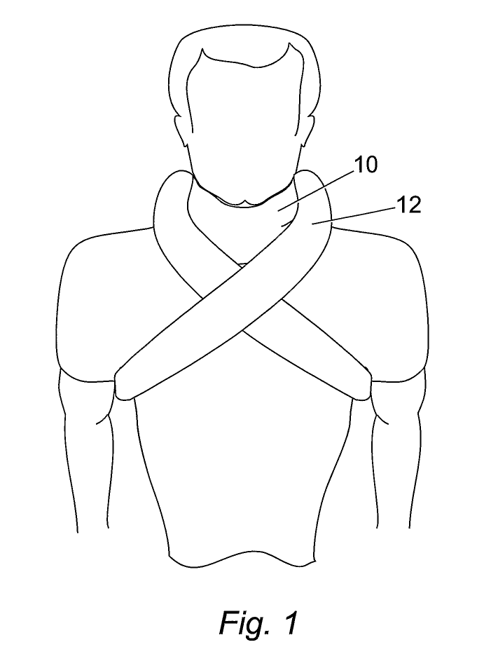

FIG. 1 is a front view of an embodiment of the apparatus, in use, wherein the elongate support device has been arranged such that it extends between a first arm and torso of the subject adjacent to an axilla, over the subject's chest, over the front of the subject's shoulder, behind the subject's neck, over the front of the subject's other shoulder, over the subject's chest and between the subject's other arm and torso adjacent to the subject's other axilla;

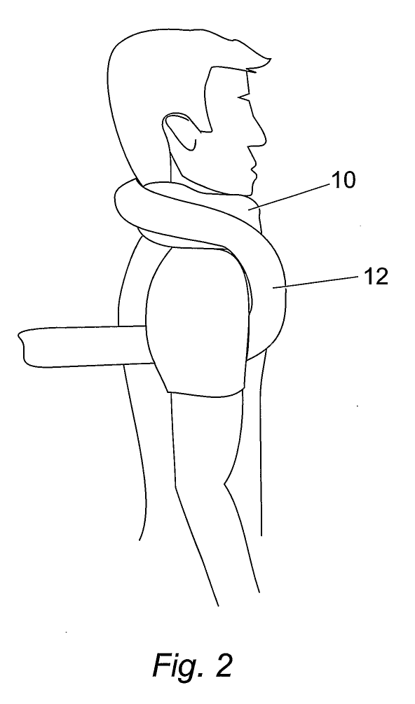

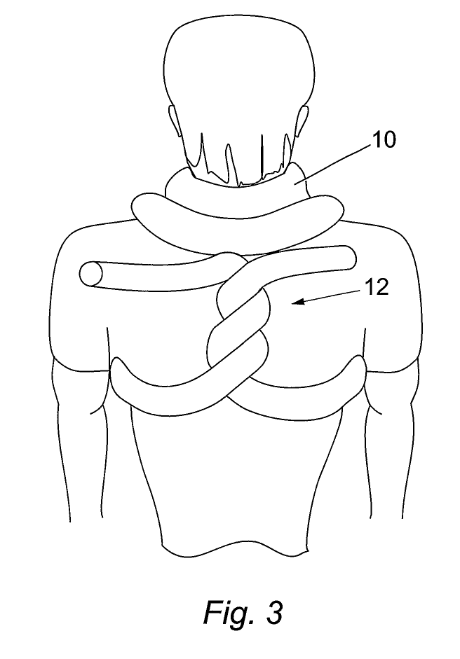

FIG. 2 is a side view of an embodiment of the apparatus, in use, wherein the elongate support device has been arranged such that it extends between a first arm and torso of the subject adjacent to an axilla, over the front of the subject's shoulder, behind the subject's neck, over the front of the subject's other shoulder and between the subject's other arm and torso adjacent to the subject's other axilla;

FIG. 3 is an illustration of an embodiment of the apparatus, as viewed from the back arranged such that it extends between a first arm and torso of the subject adjacent to an axilla, over the front of the subject's shoulder, behind the subject's neck, over the front of the subject's other shoulder and between the subject's other arm and torso adjacent to the subject's other axilla;

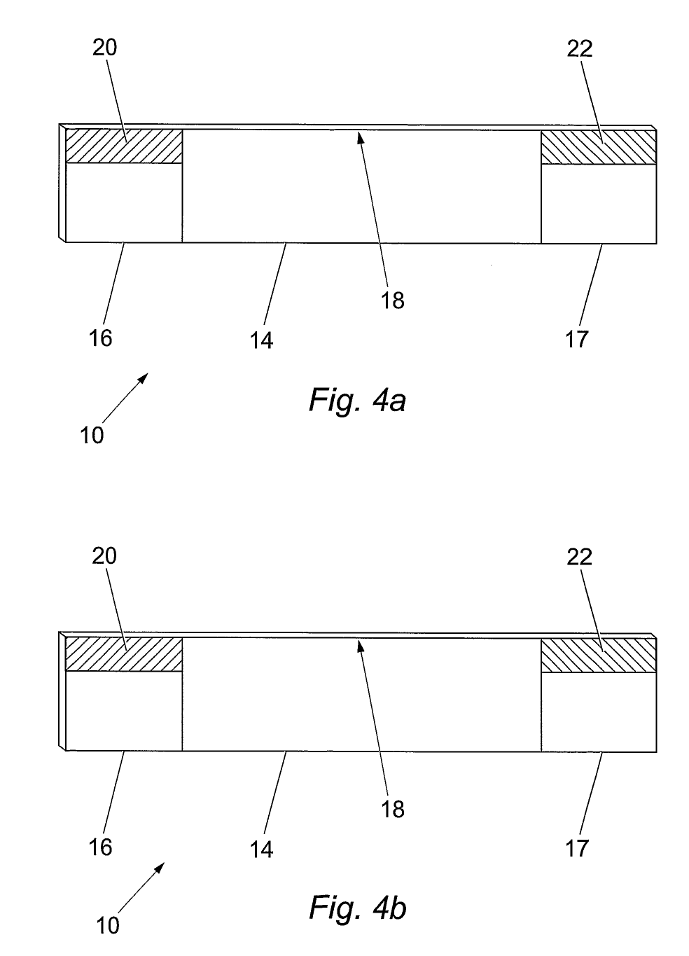

FIG. 4(a) is an illustration of a first face of an embodiment of a collar device of the invention showing fasteners which on arrangement of the device around the neck allow the device to be fastened in the arranged conformation to support the neck;

FIG. 4(b) is an illustration of the second opposite face of an embodiment of a collar device of the invention showing fasteners which on arrangement of the device around the neck allow the device to be fastened in the arranged conformation to support the neck;

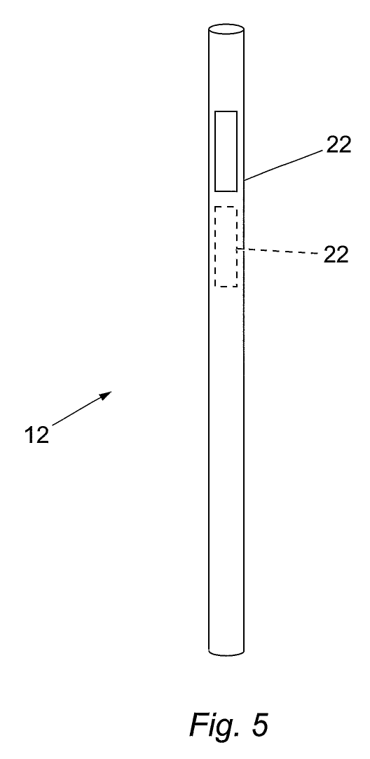

FIG. 5 is an illustration of an embodiment of an elongate support member of the invention showing a first fastener on a first side of the elongate support and a second fastener on a second side of the support such that the support can be rolled up and the first and second fasteners come into contact with each other;

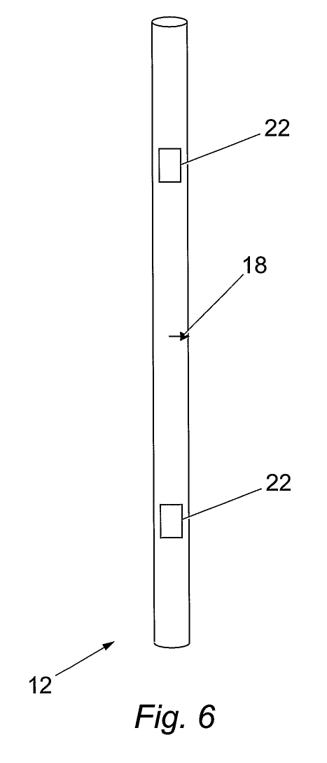

FIG. 6 is an illustration of an embodiment of an elongate support member of the invention including further fastening members, for example to attach to a leg support portion;

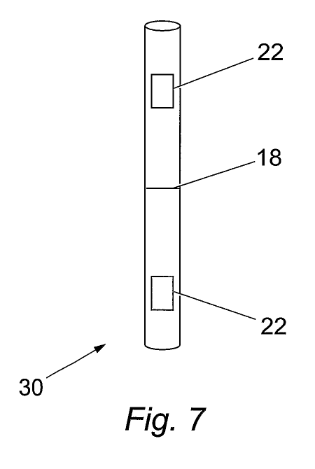

FIG. 7 is an illustration of an embodiment of a leg support member of the invention;

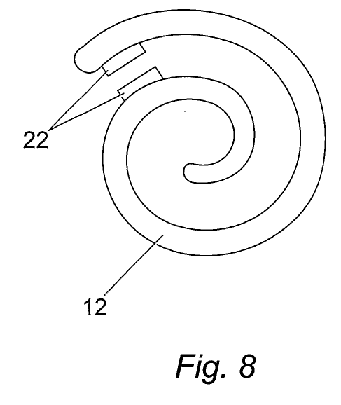

FIG. 8 is an illustration of an embodiment of the elongate support member of the invention in a rolled up state;

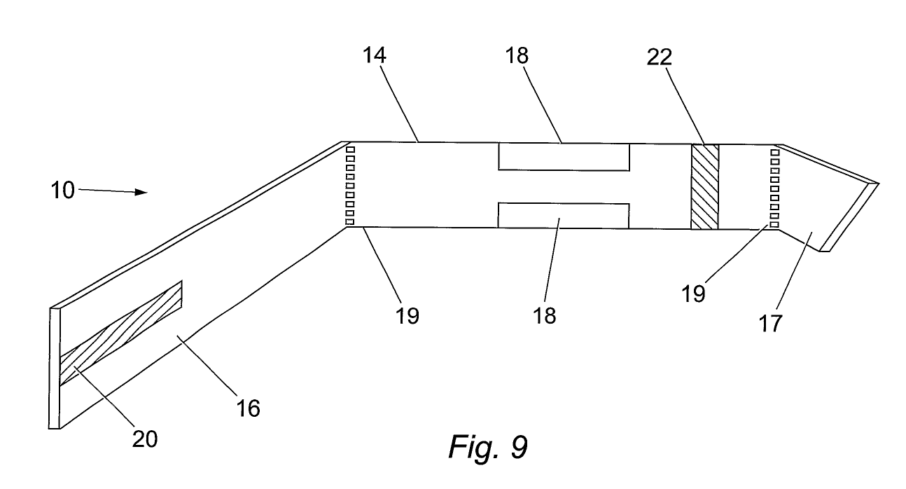

FIG. 9 is an illustration of an embodiment of collar device of the invention; and

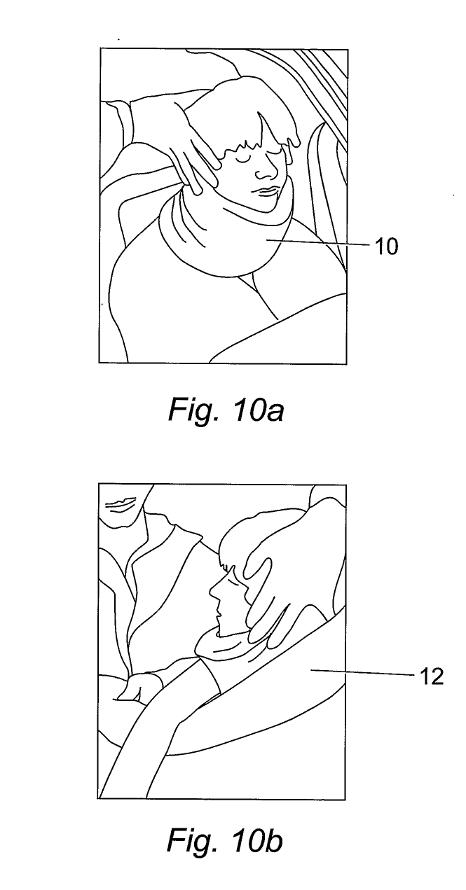

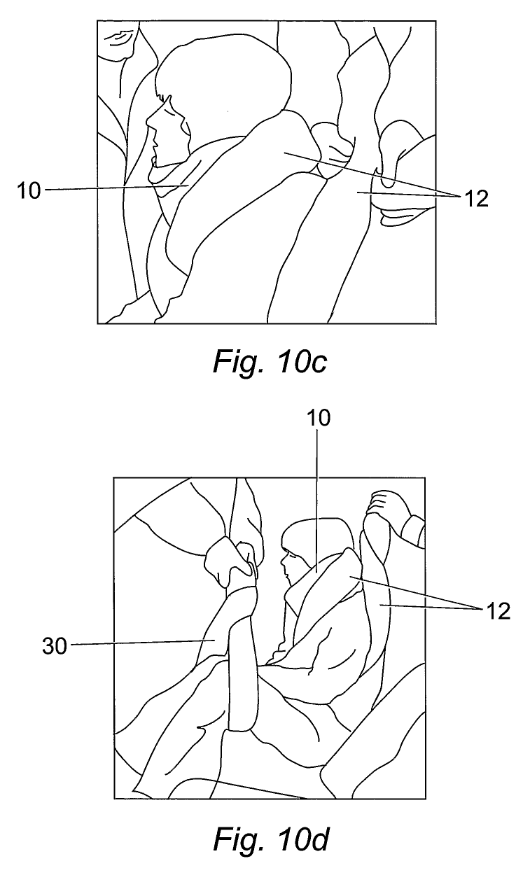

FIGS. 10a, b, c and d are illustrations of an embodiment of the apparatus, comprising a leg support, in use to extricate a subject from a vehicle.

As illustrated in FIG. 1, the spinal restraint apparatus comprises a collar device 10 and an elongate flexible support member 12. In use, the collar device is arranged around the subject's neck such that it is positioned under the mandible and extends down to the chest at the point where the clavicles meet the sternum. The collar support device can be secured around the neck, by for example VELCRO™ such that it stabilises the head and neck (minimises flexion, extension and rotation) during movement of a subject from, for example a vehicle.

The placement of the elongate support member 12, which has a load bearing capacity such that it can be used to lift the subject, on top of the collar device and tensioning of said elongate support member by twisting the support member and loading the subject's body weight onto the elongate support member (by lifting the subject) acts to compress at least part of the collar device and provide increased support to the head and neck. At no time is the airway of the subject compromised by the collar neck device.

As will be appreciated, the pliable nature of the materials from which the collar device and elongate support device are formed means that no pre-selection of the size of apparatus to be used nor dimensional measurement of the subject prior to fitting of the apparatus is required. In addition, the mouldable nature of the materials from which the collar device and elongate support device are formed means the apparatus can be used over and on top of the subject's clothing.

As illustrated in FIG. 2, in situations where it is preferable not to overlay the subject's chest when applying the elongate device, the device can be passed under the axilla and over the respective shoulder of that axilla, around the back of the subject's neck, over the other shoulder and under the axilla of that respect other shoulder.

As Illustrated in FIG. 3, tensioning of the elongate support to provide additional support to the neck by compressing at least a portion of the collar neck device can be achieved by twisting of the elongate support member behind the subject's back.

In the embodiment illustrated in FIGS. 4a and 4b, the collar device comprises a support portion 14 interposed between two connecting portions 16, 17. The regions between the support portion 14 and the connecting portions 16, 17 are thinner to allow the connecting portions to be moved in relation to the support portion more easily. This provides the collar device with additional flexibility which, in use, aids placement of the device around the neck. The support portion includes a central marker 18 to allow a user applying the collar device to a subject to rapidly determine the central point of the collar such that this point of the collar can be place under a subject's chin.

Each of the connecting portions include at least one fastening means to allow the collar device to be secured around a subject's neck once the collar device has been suitable wrapped around the neck. In the illustrated example each of the connecting portions includes two corresponding portions of VELCRO™. A first connecting portion 16 has VELCRO™ hook means 20 provided in a first face and second opposite face. A second connecting portion 17 has corresponding loop VELCRO™ means 22 provided on a first and second opposite face.

In the embodiment shown in FIGS. 4a and 4b, wherein the connecting portions are of equal length the collar neck device fastens at the back of the subject's head.

In the embodiment of the elongate support member shown in FIG. 5, the elongate member has a length of about 2.6 m such that in use it is capable of extending around a human torso between a first arm and the torso at a point adjacent to the axilla (arm pit), behind the neck and between a second arm and torso of the same human and around the back of the torso.

The elongate member can have a corresponding fastening means 20, 22, for example VELCRO™ arranged on opposite sides of the elongate support member to allow the elongate member to be rolled up along its longitudinal axis, as illustrated in FIG. 8, such that the second fastening means, for example VELCRO™ portion, becomes presented to the first fastening means, for example VELCRO™ portion, and allows the rolled up elongate member to be held in its rolled up state.

In the embodiments illustrated, the collar device 10 and elongate support member 12 are formed from knitted polyester mesh surrounded by a polyurethane coated material. The advantage of surrounding the mesh or providing a surface of polyurethane coated material to the device and members of the invention is that they can be wiped down following use and they are relatively water resistant. This provides the elongate support member with strength and flexibility. However, it will be appreciated that a wide variety of materials may be used to form the apparatus or to specifically form components of the apparatus, for example the collar device or the elongate member.

When in use, positioned around the body, it is advantageous if the elongate member and/or the collar device is able to deform and fit to the shape of the body to minimise voids between the collar device and/or support member and the back of the neck such that the elongate member and collar device act in combination to restrict the movement of the neck without compromising the airway

A marker may be provided at the centre of the elongate member or collar device, as a guide as to where, in use, the member should be placed in relation to the subject (i.e. the marker may be placed directly behind the subject's neck in the case of the elongate marker or at the subject's chin in the case of the collar device). The marker may be a strengthened portion of the device to provide additional neck support, a surface configuration or a printed or coloured portion of the components of the apparatus.

As illustrated in FIG. 6 additional or alternative fastening portions may be provided on the elongate support such that this member may be attached or fixed to a leg support.

Although not illustrated, the elongate support member 12 can optionally be provided with handles. Rescuers may then manipulate the subject to which the apparatus has been applied by grasping the handles or providing lifting straps through the handles. The handles may be provided at any point along the elongate support member. Preferably handles are provided at spaced distances from both ends of the elongate support member such that when in use the handles are provided at both sides of the subject's torso.

It is considered to be advantageous if the collar device can be applied to the subject at the side of the neck, as it is likely to be easier and possibly safer for the rescuer to gain access to the subject at this position, and obstructive features, for example, in cars, head rests are less likely to be present at the side of the subject.

As illustrated in FIG. 9, in an embodiment of a collar support portion, the support portion of collar can comprise a padded neck support 14 interposed between a first connecting portion 16 and a second connecting portion 17.

The first connecting portion includes a first end and a second end 19, wherein the second end of the connecting portion joins the first connecting portion to the neck support portion 14. A fastening device, as illustrated a VELCRO™ portion 20, adjacent to a first end of the first connecting portion is provided. This fastening device can be located at any point of the first connecting portion. When the fastening portion is provided as a VELCRO™ portion it has been found to be advantageous if this is provided in the middle of the connecting portion adjacent to the first end to provide suitable support to the neck when the collar device is positioned around the neck.

A second connecting portion has a first end and a second end, wherein the second end 19 joins the connecting portion to the neck support portion.

As illustrated by the dashed lines in FIG. 9, the region where the second ends of the connecting portions join to the support portion can be formed to allow the connecting portions to be folded back from the support portion.

The neck support portion can include a central marker 18 which, in use, functions as a chin alignment guide, wherein the user's chin is positioned in the alignment guide.

Adjacent to the end of the support portion of the collar, which joins to the second connecting portion, a VELCRO™ portion which can be joined to the VELCRO™ portion on the first connecting portion is provided. Thus, when, in use, the collar device is wrapped around a subject's neck, the collar device can be secured in position by attaching the VELCRO™ portion on the first connecting portion to the VELCRO™ of the support portion. As would be appreciated by a person of skill in the art, quick release clips or other easy to use fastenings could be provided on the collar device to allow the positioning of the collar device around the neck to be secured.

In the illustrated embodiment, the collar neck device has a flexible core formed by a semi-rigid foam covered by at least one layer of mesh. The core then has an exterior covering provided by a polyurethane coated fabric. Suitably, the core is formed from closed cell foam, around which a mesh, which can be woven or non-woven, is provided. The semi-rigid foam, for example a closed cell foam, is arranged by folding or rolling of the foam to form multiple layers, for example four layers, and then mesh is wound around the foam. The device is provided with strength in multiple directions. The mesh and foam are then encapsulated by a polyurethane coated fabric.

The placement of the fixing means as described in relation to the embodiment illustrated by FIG. 9 with the connecting portions of differing lengths is advantageous as it provides for fixation or attachment of the apparatus to a subject when the person fixing/attaching the apparatus is located at the side of the subject.

A sequence of illustrations showing the placement of the collar device and subsequent arrangement of the elongate member is illustrated in FIG. 10. In use, as shown in FIG. 10a, the subject's head is held by a rescuer in a neutral position and the collar neck support device is wrapped around the subject's neck between the mandible and the chest to minimise forward movement of the head. The collar can then be secured in place using fastening means, for example VELCRO™. It will be appreciated that any other suitable fastening means, for example quick release buckles, straps or the like could also or alternatively be used. The fastening means can be located at any point around the subject's neck, but are most advantageously provided at a side of the subject's neck and this is likely to be clear of obstructions, for example head rests. A marker provided on the collar can indicate to a rescuer which portion of the collar device should be placed, for example, under the subject's chin such that the device can be positioned correctly. As shown in FIG. 10b, following application of the collar device, in use, the elongate support member 12 is positioned such that it extends between a first arm and torso of the subject adjacent to an axilla, over the front of the subject's shoulder, behind the subject's neck, over the front of the subject's other shoulder and between the other subject's arm and torso adjacent to the subject's other axilla. In this position the elongate support member can overly the collar device and provide further resistance to movement of the head from the neutral pressure.

In one embodiment of the method, as shown in FIG. 10b, in the step of positioning the flexible elongate support member, the elongate support member extends between a first arm and torso of the subject adjacent to an axilla, crosses the subject's chest and passes over the front of the subject's shoulder opposite to the axilla under which the elongate support member extends, passes behind the subject's neck, over the front of the subject's other shoulder, crosses the subject's chest and passes and between the subject's other arm and torso adjacent to the subject's other axilla.

In an alternative embodiment of the method, in the step of positioning the elongate support member, the elongate support member is not passed over the subject's chest, but extends between a first arm and torso of the subject adjacent to an axilla, passes over the front of the subject's shoulder above the axilla under which the elongate support member extends, passes behind the subject's neck, over the front of the subject's other shoulder and between the subject's other arm and torso adjacent to the subject's other axilla. This is illustrated in FIG. 2. A marker provided on the elongate member can guide the rescuer as to the portion of the elongate member which should be placed behind the subject's neck.

As illustrated in FIG. 10c, once the elongate device has been passed around the back of the subject's neck and under each axilla, the elongate member 12 can be pulled by a rescuer behind the subject's back to apply tension to the elongate member. In addition, a first end of the elongate member can be twisted around the second end of the elongate member to increase this tension provided. Applying this tension can be advantageous to increase the support provided to the head to restrain the head in, or close to, a neutral position. During extrication a further support member may be provided to the subject's legs just above the knees of the subject.

When appropriate, once the collar device and elongate support member have been applied to the subject, a rescuer can grasp the elongate member to provide tension to each of the ends of the elongate support member to suitably hold the subject's head and neck and lift the subject away from the location of danger, for example extricate the subject from a vehicle. As illustrated in FIG. 10d, a second rescuer can lift the subject's legs using the leg support at the same time as the elongate member is lifted whilst the subject is moved.

A significant advantage of the extrication device of the present application is that it can be rapidly provided to a subject with potential spinal cord injury who requires emergency extrication. It does not require pre-sizing of the component parts relative to the subject, and does not require careful adjustment once applied to the subject.

Further, the relatively small dimensions of the device and its flexibility means that it does not interfere with access to the subject, nor will it be difficult to store the restraint device in rescue vehicles.

Modifications and improvements may be made to the above without departing from the scope of the present invention.

What is claimed is:

1. A spinal restraint apparatus for emergency extrication comprising:
(i) an elongate flexible support member of length in the range 0.5 m to 5.5 m; and
(ii) a collar neck support device.

2. The spinal restraint apparatus of claim 1 which, when the elongate flexible support member and collar neck support device are used in combination, supports the head and neck within 10 degrees of a neutral position.

3. The spinal restraint apparatus of claim 1 wherein the collar neck support device is formed from pliable and/or mouldable material such that the collar neck support device is capable of being wrapped around a subject's neck such that it moulds to the shape of the neck and fits between the subject's mandible of the jaw and the top of the subject's clavicles where they meet the sternum.

4. The spinal restraint apparatus of claim 1 wherein said collar neck support device includes fastening means, selected from the group comprising, VELCRO™, quick release clips, straps and buckles, hooks and eyelets, and tying straps to hold the collar neck support device around a subject's neck.

5. The spinal restraint apparatus of claim 1 wherein the elongate support member has a length in the range 1 m to 4 m.

6. The spinal restraint apparatus of claim 1 wherein the elongate support member comprises a handle.

7. The spinal restraint apparatus of claim 1 further comprising a leg support portion.

8. The spinal restraint apparatus of claim 7 wherein the leg support portion has a width in the range 4 cm to 15 cm.

9. The spinal restraint apparatus of claim 7 wherein the leg support portion has a length in the range of 1 m to 5 m.

10. The spinal restraint apparatus of claim 1 wherein the collar neck support device is of length in the range 0.4 m to 1.2 m.

11. The spinal restraint apparatus of claim 1 wherein the collar neck support device has a width in the range 10 cm to 20 cm.

12. The spinal restraint apparatus of claim 1 wherein the collar neck support device has a thickness in the range 1.5 cm to 5 cm.

13. The spinal restraint apparatus of claim 1 wherein the elongate flexible support member has a width in the range 4 cm to 15 cm.

14. The spinal restraint apparatus of claim 1 wherein the elongate flexible support member can lift a load of at least 150 kg.

15. The spinal restraint apparatus of claim 1 wherein the elongate flexible support member or the collar neck support device is formed from a polyester knitted fabric encapsulated by a polyurethane coated outer fabric.

16. The spinal restraint apparatus of claim 1 wherein the collar neck support device comprises a collar support portion interposed between two connecting portions wherein at least one of the connecting portions includes fastening means, selected from the group comprising, VELCRO™, quick release clips, straps and buckles, hooks and eyelets, and tying straps to hold the collar neck support device around a subject's neck.

17. The spinal restraint apparatus of claim 16 wherein the collar support portion is of length in the range 30 cm to 50 cm and the connecting portions are in the range 15 cm to 25 cm in length.

18. The spinal restraint apparatus of claim 16 wherein the collar support portion is of thickness in the range 1.5 cm to 5 cm and the connecting portions are of thickness in the range 1 mm to 10 mm.

19. The spinal restraint apparatus of claim 16 wherein the collar support portion is about 40 cm in length, a first connecting portion is about 7 cm in length and a second connecting portion is about 25 cm in length, wherein the collar support portion and the second connecting portion are provided with fastening means to secure the collar neck support device around a neck of a subject.

20. A method of restricting movement of a subject's head, the method comprising:
(i) providing a collar neck support device to a subject,
(ii) arranging said collar neck support device around the neck of the subject to support the head in at least the anterior position,
(iii) providing a flexible elongate support member to a subject,
(iv) positioning the flexible elongate support member such that it extends between a first arm and torso of the subject adjacent to an axilla, over the front of the subject's shoulder, behind the subject's neck, over the front of the subject's other shoulder and between the subject's other arm and torso adjacent to the subject's other axilla, and
(v) tensioning the elongate support member to restrict movement of the head of the subject in at least a posterior direction.

21. A method of moving a subject comprising:
(i) providing a collar neck support device to a subject,
(ii) arranging said collar neck support device around the neck of the subject to support the head in at least the anterior direction,
(iii) providing a flexible elongate support member to a subject,
(iv) positioning the flexible elongate support member such that it extends between a first arm and torso of the subject adjacent to an axilla, over the front of the subject's shoulder, behind the subject's neck, over the front of the subject's other shoulder and between the subject's other arm and torso adjacent to the subject's other axilla,
(v) tensioning the elongate support member to restrict movement of the head of the subject in at least a posterior direction, and
(vi) moving the subject.

22. A method of claim 20 further comprising providing at least one leg support to the subject.

23. A method of claims 20 wherein in the positioning the flexible elongate support member, the elongate support member extends between a first arm and torso of the subject adjacent to an axilla, crosses the subject's chest and passes over the front of the subject's shoulder opposite to the axilla under which the elongate support member extends, passes behind the subject's neck, over the front of the subject's other shoulder, crosses the subject's chest and passes between the subject's other arm and torso adjacent to the subject's other axilla.

24.-26. (canceled)
